SEQUENCE LISTING (35 sequences)

SEQ ID NO: 1 - protein, 7 aa, Artificial sequence
  Synthetic
ENLYFQG

SEQ ID NO: 2 - protein, 7 aa, Artificial sequence
  Synthetic
ENLYFQS

SEQ ID NO: 3 - protein, 3 aa, Artificial sequence
  Synthetic
GGG

SEQ ID NO: 4 - protein, 5 aa, Artificial sequence
  Synthetic
GGGGS

SEQ ID NO: 5 - protein, 4 aa, Artificial sequence
  Synthetic
GGSG

SEQ ID NO: 6 - protein, 4 aa, Artificial sequence
  Synthetic
SGGG

SEQ ID NO: 7 - protein, 4 aa, Artificial sequence
  Synthetic
GSGS

SEQ ID NO: 8 - protein, 6 aa, Artificial sequence
  Synthetic
GSGSGS

SEQ ID NO: 9 - protein, 8 aa, Artificial sequence
  Synthetic
GSGSGSGS

SEQ ID NO: 10 - protein, 10 aa, Artificial sequence
  Synthetic
GSGSGSGSGS

SEQ ID NO: 11 - protein, 12 aa, Artificial sequence
  Synthetic
GSGSGSGSGSGS

SEQ ID NO: 12 - protein, 6 aa, Artificial sequence
  Synthetic
GGSGGS

SEQ ID NO: 13 - protein, 9 aa, Artificial sequence
  Synthetic
GGSGGSGGS

SEQ ID NO: 14 - protein, 12 aa, Artificial sequence
  Synthetic
GGSGGSGGSGGS

SEQ ID NO: 15 - protein, 4 aa, Artificial sequence
  Synthetic
GGSG

SEQ ID NO: 16 - protein, 8 aa, Artificial sequence
  Synthetic
GGSGGGSG

SEQ ID NO: 17 - protein, 12 aa, Artificial sequence
  Synthetic
GGSGGGSGGGSG

SEQ ID NO: 18 - protein, 5 aa, Artificial sequence
  Synthetic
GGGGS

SEQ ID NO: 19 - protein, 15 aa, Artificial sequence
  Synthetic
GGGGSGGGGSGGGGS

SEQ ID NO: 20 - protein, 18 aa, Artificial sequence
  Synthetic
GNGNGNGNGNGNGNGNGN

SEQ ID NO: 21 - protein, 11 aa, Artificial sequence
  Synthetic
GNGNGNGNGTG

SEQ ID NO: 22 - protein, 9 aa, Artificial sequence
  Synthetic
GGGGAGGGG

SEQ ID NO: 23 - protein, 10 aa, Artificial sequence
  Synthetic
GENLYFQSGG

SEQ ID NO: 24 - protein, 8 aa, Artificial sequence
  Synthetic
SACYCELS

SEQ ID NO: 25 - protein, 5 aa, Artificial sequence
  Synthetic
RSIAT

SEQ ID NO: 26 - protein, 17 aa, Artificial sequence
  Synthetic
RPACKIPNDLKQKVMNH

SEQ ID NO: 27 - protein, 36 aa, Artificial sequence
  Synthetic
GGSAGGSGSGSSGGSSGASGTGTAGGTGSGSGTGSG

SEQ ID NO: 28 - protein, 17 aa, Artificial sequence
  Synthetic
AAANSSIDLISVPVDSR

SEQ ID NO: 29 - protein, 36 aa, Artificial sequence
  Synthetic
GGSGGGSEGGGSEGGGSEGGGSEGGGSEGGGSGGGS

SEQ ID NO: 30 - protein, 18 aa, Artificial sequence
  Synthetic
GNGNGNGNGNGNGNGNGN

SEQ ID NO: 31 - protein, 11 aa, Artificial sequence
  Synthetic
GNGNGNGNGTG

SEQ ID NO: 32 - protein, 10 aa, Artificial sequence
  Synthetic
GRKKRRQRRR

SEQ ID NO: 33 - protein, 7 aa, Artificial sequence
  Synthetic
PKKKRKV

SEQ ID NO: 34 - protein, 20 aa, Artificial sequence
  Synthetic
AVKRPAATKKAGQAKKKKLD

SEQ ID NO: 35 - protein, 9 aa, Artificial sequence
  Synthetic
PAAKRVKLD

(19) United States
(12) Patent Application Publication
(10) Pub. No.: US 2022/0404337 A1
(43) Pub. Date: Dec. 22, 2022

(54) REAL-TIME G-PROTEIN COUPLED RECEPTOR (GPCR) LINKED BIOLUMINESCENT SENSING OF BIOLOGICAL TARGETS AND PROCESSES

(71) Applicant: Massachusetts Institute of Technology, Cambridge, MA (US)

(72) Inventors: Frances Nargi, Littleton, MA (US); Scott Thomas Wick, Ayer, MA (US); Deepak Mishra, Cambridge, MA (US)

(73) Assignee: Massachusetts Institute of Technology, Cambridge, MA (US)

(21) Appl. No.: 17/726,326

(22) Filed: Apr. 21, 2022

Related U.S. Application Data
(60) Provisional application No. 63/212,902, filed on Jun. 21, 2021.

(51) Int. Cl. G01N 33/50; C12N 15/62; C12N 9/02; C07K 14/72; C07K 14/66

(52) U.S. Cl. CPC G01N 33/5023; C12N 15/625; C12N 9/0071; C12Y 114/14003; C07K 14/723; C07K 14/721; C07K 14/66; G01N 2333/726; C07K 2319/03; C07K 2319/50; C07K 2319/09

(57) ABSTRACT

The invention relates to compositions and methods for making and use of a real-time cellular sensor. Components of a multipart enzyme are sequestered in different cellular compartments and only come together after receptor activation; a pool of substrate is made available in the cell to ensure real-time enzymatic output.

### RELATED APPLICATION

This application claims the benefit under 35 U.S.C. § 119(e) of U.S. provisional application number 63/212902, filed Jun. 21, 2021, which is incorporated by reference herein in its entirety.

### FEDERALLY SPONSORED RESEARCH

This invention was made with government support under FA8702-15-D-0001 awarded by the U.S. Air Force. The government has certain rights in the invention.

### BACKGROUND OF INVENTION

Biological signaling molecules such as proteins, hormones, and neurotransmitters can serve as important markers for monitoring health, assessing the effects of therapeutics or environmental stressors, diagnosing disease, and guiding medical treatment. There are a number of available sensor systems for sensitive and specific measurement of molecular biomarkers. These generally require biological fluid collection, processing, and analysis steps that take days to weeks to obtain and typically involve analyte measurement at a single time point. Monitoring biological molecules in real-time on a continuous or high frequency basis would enable adverse trends to be identified quickly, drug or stressor responses to be tracked dynamically, and molecular cycles to be characterized. Research and development into real-time sensors for implanted or non-invasive biomarker monitoring have focused on electrochemical, optical, mass spectrometric, or surface-plasmon resonance approaches. These have created new capabilities but rely on expensive detection equipment, have limited specificity, and high reagent costs.

### SUMMARY OF INVENTION

The instant disclosure relates to a recombinant cell comprising exogenous, constitutively-expressed nucleic acids that express protein comprising an endogenous biological circuit. This circuit detects, if present, the presence of a molecule of interest, which activates a receptor and that receptor's downstream signaling pathways, generating a detectable enzymatic output. Because the components of the circuit are constitutively expressed, detection of a molecule of interest does not require waiting for transcription or translation.

In some aspects, the invention is a biosensor device. The device has a housing comprising a surface for accepting a ligand, a recombinant cell within the housing having a biological circuit, wherein the biological circuit comprises a multi-part enzyme, wherein two or more parts of the multi-part enzyme have spatially distinct localizations and wherein when the recombinant cell is exposed to the ligand, the two or more parts of the multi-part enzyme are triggered to co-localize and interact with a substrate molecule to generate a measurable immediate read-out bioluminescent signal, and a surface for observing the bioluminescent signal.

In some embodiments the multi-part enzyme is lux and wherein the two or more parts of the multi-part enzyme are luxA and luxB. In some embodiments the recombinant cell has a cell surface receptor for the ligand. In some embodiments the cell surface receptor comprises a GPCR complex. In some embodiments the GPCR complex is linked to luxA. In some embodiments the GPCR complex is linked to luxA through a linker having a protease cleavage site. In some embodiments the protease cleavage site is linked to an anchor site sequence which is linked to the luxA. The anchor site is a membrane, including intracellular facing plasma membrane or intracellular membrane locations, such as a nuclear-localization sequence. In some embodiments the GPCR is tethered to the plasma membrane. In some embodiments the luxB is localized in the nucleus, mitochondria, endoplasmic reticulum, or other cell membrane-integrated protein.

In other embodiments the biosensor device is a wearable device. In some embodiments the device includes a flexible mount configured, during use, to be affixed to a user's body, the mount comprising a body attachment means for affixing said mount to the user's body.

In other aspect the invention is a method for sensing a ligand in real-time, by exposing a putative ligand containing material to a biosensor device, examining the biosensor device for the presence of a bioluminescent signal immediately upon exposure to the putative ligand containing material, wherein the presence of a bioluminescent signal in the biosensor device indicates the presence of the ligand in the material in real-time, wherein the biosensor device comprises a recombinant cell having a biological circuit, wherein the biological circuit comprises a multi-part enzyme, wherein two or more parts of the multi-part enzyme have spatially distinct localizations and wherein when the recombinant cell is exposed to the ligand, the two or more parts of the multi-part enzyme are triggered to co-localize and interact with a substrate molecule to generate a measurable immediate read-out bioluminescent signal indicative of the presence of the ligand.

In some embodiments the biosensor device is any of the devices described herein.

In an aspect, the instant disclosure relates to a recombinant cell. In some embodiments, the cell comprises a constitutively expressed exogenous nucleic acid encoding one or more proteins that produce a measurable enzymatic output, each protein comprising at least two different components, a first component anchored to a nuclear-localized protein and a second component tethered to a plasma membrane-bound receptor by a linker that comprises a protease cleavage site, wherein at least one of each of the at least two components comes together to produce enzymatic output, and wherein binding of a ligand to the plasma-membrane-bound receptor results in activation of the receptor, cleavage of the linker by a protease, and release of the second component tethered to the receptor. In some embodiments, the cell also comprises a constitutively expressed exogenous nucleic acid encoding an enzyme complex that synthesizes a substrate for enzymatic output of the different polypeptide components.

In an embodiment, the plasma membrane protein is a G Protein-coupled receptor (GPCR). In an embodiment, the linker further comprises a spacer sequence. In an embodiment, the protease is a recombinant protease.

In an embodiment, the nucleic acid encodes luxABCDEfrp and the measurable enzymatic output is bioluminescence. In an embodiment, the second component is luxA and the first component is luxB.

In some embodiments, the recombinant cell comprises a constitutively active exogenous nucleic acid encoding: (a) a protein complex producing a measurable enzymatic output, the protein complex comprising a first component anchored to a nuclear-localized protein and a second component tethered to a plasma membrane-bound receptor by a linker that comprises a protease cleavage site; and (b) an enzyme complex that produces a substrate of the protein complex, wherein binding of a ligand to the plasma-membrane-bound receptor results in activation of the receptor, cleavage of the linker by a protease, and release of the second component tethered to the receptor.

In some embodiments, the recombinant cell comprises a constitutively active exogenous nucleic acid encoding: (a) a recombinant sensor, comprising: (i) a ligand binding domain; (ii) a transmembrane domain; (iii) a protease cleavage site; (iv) an anchor site sequence such as a nuclear-localization sequence; and (v) a first component of a two-part enzyme, wherein the protease cleavage site is between the transmembrane domain and the an anchor site sequence; (b) a nuclear receiver comprising: (i) a second component of the two-part enzyme; (ii) a nuclear-localized protein; and (c) an enzyme complex that produces a substrate, a co-factor, or both a substrate and a co-factor.

In an embodiment, the recombinant sensor further comprises a spacer sequence between the transmembrane domain and the protease cleavage site.

In another aspect, the instant disclosure relates to a method of detecting receptor activation. In some embodiments, the method comprises: (a) introducing to a cell a constitutively expressed nucleic acids encoding: (i) one or more proteins that produce a measurable enzymatic output, each protein comprising at least two different components, a first component anchored to a nuclear-localized protein and a second component tethered to a plasma membrane-bound receptor by a linker that comprises a protease cleavage site, wherein at least one of each of the at least two components comes together to produce enzymatic output; and (ii) an enzyme complex that synthesizes a substrate for enzymatic output of the different polypeptide components, wherein, binding of a ligand to the plasma-membrane-bound receptor results in activation of the receptor, cleavage of the linker by a protease, and release of the second component tethered to the receptor; (iii) measuring levels of enzymatic output, wherein detectable levels of enzymatic output indicate receptor activation.

In some embodiments, the method, comprises: (a) providing a recombinant cell disclosed herein; (b) maintaining the cell under suitable conditions; and (c) measuring levels of enzymatic output, wherein detectable levels of enzymatic output indicate receptor activation.

In some embodiments, the method further comprises designing a treatment plan based upon the measured output.

In some embodiments, the codon-optimized nucleic acid encodes luxABCDEfrp and the measurable enzymatic output is bioluminescence. In some embodiments, the second component is luxA and the first component is luxB. In some embodiments, the plasma membrane-bound receptor is a GPCR. In some embodiments, the protease is a recombinant protease.

In another aspect, the instant disclosure relates to an isolated nucleic acid encoding: (a) a constitutive promoter coupled to genes encoding (b) one or more proteins capable of producing a measurable enzymatic output, each protein comprising at least two different components, a first component anchored to a nuclear-localized protein and a second component tethered to a plasma membrane-bound receptor by a linker that comprises a protease cleavage site, wherein at least one of each of the at least two components comes together to produce enzymatic output, and (c) an enzyme complex synthesizing a substrate for the enzymatic output of the different components.

In another aspect, the instant disclosure relates to a biological circuit, comprising: (a) a recombinant cellular sensor, comprising a membrane-bound receptor protein, translationally fused to a first portion of a multi-part enzyme by a protease cleavable linker, wherein the first portion of the multi-part enzyme further comprises a nuclear localization sequence; (b) a second portion of the multi-part enzyme where the second portion of the multi-part enzyme is confined to the nucleus, mitochondria, endoplasmic reticulum, or other cell membrane-integrated protein; and (c) a pool of substrate for the multi-part enzyme, wherein binding of a ligand to the plasma-membrane-bound receptor results in activation of the membrane-bound receptor, cleavage of the linker at the protease cleavage site, and the first portion and the second portion of the multi-part enzyme come together, processing the pool of substrate molecules to generate a measurable signal.

In some embodiments, the biological circuit further comprises enzymes produced by exogenous nucleic acids for producing the pool of substrate.

### BRIEF DESCRIPTION OF DRAWINGS

FIG. 1. Schematic depicting design and functionality of the receptor—luxA domain within the receptor activation sensing system. Component domains are labeled and corresponding function can be found in the text.

FIG. 2. Schematic depicting biochemical concept of receptor linked, lux-based ligand detection. Receptor-linked components are used to report on ligands interacting with GPCRs (G-coupled protein receptors). Unlike previous designs, luxCDEfrp genes constitutively synthesize bacterial luciferin (myristic aldehyde) and allow for immediate sensing. (A) Unstimulated cell with luxA and luxB segregated (no ligand present, no bioluminescence) (B) Ligand binding to GPCR stimulates signal transduction pathway resulting in luxAB bioluminescence production in the nucleus.

FIGS. 3A-3B. (3A) Signal to background ratio for stimulated receptor (Adrenaline +ADRB2) versus unstimulated cells for varying ratios of the 2 transfected DNAs. Receptor DNA composed of sensor ADRB2-luxA+LAP2-luxB fusion. Arr2-TEVp is signal transduction trigger DNA, co-expressed with luminescence “fuel” genes (luxCDEfrp). (3B) Graphical representation of tabular DNA. A ratio of between 3:1 and 5:1 sensor-to-fuel/Arr2-TEVp DNA provided best signal to background ratio under the experimental conditions tested.

### DETAILED DESCRIPTION OF INVENTION

Recently there has been an increased focus on the development of living cells to specifically sense biomolecules and transduce that recognition into a detectable signal. A number of cell-based sensor platforms able to respond to important biological molecules exist but many are built for use in in vitro endpoint assays and link receptor recognition to transcription of a reporter protein that produces measurable output. These cell-based sensors can be highly specific for biomolecules, but the transcription-based systems are too slow for most real-time applications. Fluorescent or luminescent reporter output systems can deliver more rapid results compared to reporter gene systems, but using known luminescent systems requires an exogenous supply of an expensive, labile substrate that needs to cross the cell membrane and avoid clearance/metabolism to accumulate at sufficient concentrations at the site of analysis.

The instant disclosure relates to an engineered mammalian cell sensor capable of use in real-time detection of biomarkers, in a cell, for example, a cell line, through introduction of exogenous sensor genes. This enables efficient testing of different engineered constructs and rapid assessment of sensor performance, sensitivity, and stability. The system can be used with diverse, physiologically relevant cell types. These cells can be used for in vivo monitoring of samples such as body fluids, gasses or solids. Alternatively they can be used in a living organism, for instance, in the form of an implant to as a wearable device. In some embodiments, constructing the cell sensor comprises introducing exogenous components into a cell, to establish an endogenous biological signal amplification system that produces a change in enzymatic output when the cell comprising such an amplification system recognizes a molecule of interest, also referred to herein as a ligand. An exogenous component is one which is introduced into the cell. An exogenous gene for instance refers to a gene that is introduced into the cell. It may be a gene that does or does not exist in a naturally occurring version of the cell. Recognition can be binding, hybridizing, or interacting with a molecule of interest. In some embodiments, a molecule of interest is a molecule that activates a pathway of interest. In some embodiments, a pathway of interest is or relates to a biomarker for health or disease in a subject.

The instant disclosure relates to a recombinant cell, comprising a constitutively expressed exogenous nucleic acid that encodes one or more proteins that produce a detectable enzymatic output. In an embodiment, detectable output is measurable output. In an embodiment, detectable output is observable output. In some embodiments, the measurable enzymatic output is bioluminescence. In some embodiments, the one or more proteins are luciferase proteins or luciferase protein subunits. In an embodiment, the proteins are luxA and luxB from Photorhabdus luminescens.

The protein or proteins capable of producing a measurable output comprise at least two different components sequestered in different cellular locations. For instance they may be: a first component sequestered in the nucleus, mitochondria, endoplasmic reticulum, or other cell membrane-integrated protein and a second component tethered to a plasma membrane-bound receptor by a linker that comprises a cleavage site such as a protease cleavage site. The first component and the second component are capable of producing detectable levels of the desired enzymatic output when working in concert, which requires close physical proximity.

Binding of the cognate ligand to the membrane-bound receptor results in protease cleavage of the linker that comprises a protease cleavage site, freeing the component tethered to a plasma membrane-bound receptor, which then is available to travel sufficiently close to the first component. The first and second components generate enzymatic output once they are sufficiently close together. In an embodiment, sufficient proximity or closeness means the two components are capable of binding to one another. In an embodiment, sufficient proximity or closeness means that the two components are vicinal to one another. In an embodiment, sufficient proximity or closeness entails the formation of a heterodimer. Not wishing to be bound by theory, this enzymatic output is generated by conversion of a substrate that is constitutively present in the cell, which means that the limiting factor of detectable enzymatic output is the co-localization of the first and the second components. In the present method, receptor activation results in real-time generation of measurable enzymatic output without having to wait for transcription or translation to occur, as is the case for reporter-gene-mediated signaling methods for detecting pathway activation.

In an embodiment, real-time generation of signal indicates measurable enzymatic output within seconds or minutes. In some embodiments, enzymatic output remains measurable for seconds, minutes, hours, days, or weeks.

In another aspect, the instant disclosure relates to a recombinant cell, comprising a constitutively active exogenous nucleic acid that encodes: (a) a recombinant sensor, comprising: (i) a ligand binding domain; (ii) a transmembrane domain; (iii) a protease cleavage site; (iv) an anchor site sequence such as a nuclear-localization sequence; and (v) a first component of a two-part enzyme, wherein the protease cleavage site is between the transmembrane domain and the an anchor site sequence; (b) a nuclear receiver comprising: (i) a second component of the two-part enzyme; (ii) a nuclear-localized protein; and (c) an enzyme complex that produces a substrate, a co-factor, or both a substrate and a co-factor.

In an embodiment, constitutive activity indicates continuous expression of the gene products of a nucleic acid. Not wishing to be bound by theory, this is achieved by selection of an appropriate promoter to direct expression of a particular gene. Constitutively active promoters are known to those of skill in the art, as are the properties of a range of promoters to achieve desired expression levels. In an embodiment, a constitutive promoter is the Cytomegalovirus (CMV) promoter.

The composition disclosed herein, in aspects, is a recombinant receptor protein. The recombinant receptor protein in some embodiments comprises a ligand binding domain, a cleavable linker such as a protease cleavable linker, and part of a multipart enzyme. In some embodiments, a receptor protein is a membrane-bound receptor and the recombinant receptor protein further comprises a transmembrane domain.

In some embodiments, the plasma membrane protein is a G Protein-coupled receptor (GPCR). A G-protein linked receptor (GPCR) is a member of a large family of signaling proteins found in most higher eukaryotes. GPCRs are sometimes referred to as heptahelical receptors or 7-TM receptor (Kroeze, W.K., et al, Nat Struct Mol Biol 2015). Eight different classes of GPCRs exist within humans, each differing on which G-protein couples to the receptor, and what the final signal transduction output response is made by the cell.

Not wishing to be bound by theory, GPCRs are phosphorylated by kinases (GRK); phosphorylation recruits arrestin. The phosphorylated C-tail of GPCR thought to displace arrestin c-tail and activate arrestin. GRKs are recruited to the site of an active GPCR by the production of Gβγ from an activated G protein (Gα dissociates from Gβγ). GPCR binding to a ligand stabilizes an active conformation (one able to interact with Gα).

Upon ligand binding, the GPCR causes a conformational change of Gα, causing Gβγ to dissociate, recruiting a GRK to the GPCR. The GPCR is phosphorylated by a GRK, which recruits arrestin to bind the GPCR and become activated, blocking GPCR activity.

A linker may be used to join the second component of the multipart enzyme to the receptor protein. In some embodiments, the linker comprises a protease cleavage site. In some embodiments, the protease cleavage site is ENLYFQG (SEQ ID NO. 1) or ENLYFQS (SEQ ID NO. 2),

In some embodiments, the linker further comprises a spacer sequence. In the present disclosure, a spacer can be a protein domain, such as ICAM1 or a splice variant thereof as described above, or a peptide spacer of varying lengths. Suitable peptide spacers that are known in the art include, for example, peptide linkers containing flexible amino acid residues such as glycine and serine. In certain embodiments, a spacer can contain peptide linker motifs, e.g., multiple or repeating motifs, of GS, GGS, GGG (SEQ ID NO: 3), GGGGS (SEQ ID NO: 4), GGSG (SEQ ID NO: 5), or SGGG (SEQ ID NO: 6). In certain embodiments, a spacer can contain 2 to 12 amino acids including motifs of GS, e.g., GS, GSGS (SEQ ID NO: 7), GSGSGS (SEQ ID NO: 8), GSGSGSGS (SEQ ID NO: 9), GSGSGSGSGS (SEQ ID NO: 10), or GSGSGSGSGSGS (SEQ ID NO: 11). In certain other embodiments, a spacer can contain 3 to 12 amino acids including motifs of GGS, e.g., GGS, GGSGGS (SEQ ID NO: 12), GGSGGSGGS (SEQ ID NO: 13), and GGSGGSGGSGGS (SEQ ID NO: 14). In yet other embodiments, a spacer can contain 4 to 12 amino acids including motifs of GGSG, e.g., GGSG (SEQ ID NO: 15), GGSGGGSG (SEQ ID NO: 16), or GGSGGGSGGGSG (SEQ ID NO: 17). In other embodiments, a spacer can contain motifs of GGGGS (SEQ ID NO: 18), e.g., GGGGSGGGGSGGGGS (SEQ ID NO: 19). In other embodiments, a spacer can also contain amino acids other than glycine and serine, e.g., multiple or repeating motifs of GN (e.g., GNGNGNGNGNGNGNGNGN (SEQ ID NO: 20)), GNGNGNGNGTG (SEQ ID NO: 21), GGGGAGGGG (SEQ ID NO: 22), GENLYFQSGG (SEQ ID NO: 23), SACYCELS (SEQ ID NO: 24), RSIAT (SEQ ID NO: 25), RPACKIPNDLKQKVMNH (SEQ ID NO: 26), GGSAGGSGSGSSGGSSGASGTGTAGGTGSGSGTGSG (SEQ ID NO: 27), AAANSSIDLISVPVDSR (SEQ ID NO: 28), or GGSGGGSEGGGSEGGGSEGGGSEGGGSEGGGSGGGS (SEQ ID NO: 29). The length of the peptide spacer and the amino acids used can be adjusted depending on the two protein domains involved and the degree of flexibility desired in the final protein construct. The length of the spacer can be adjusted to ensure proper protein folding and avoid aggregate formation. In certain embodiments of the invention, the linker contains the amino acid sequence GNGNGNGNGNGNGNGNGN (SEQ ID NO: 30) or GNGNGNGNGTG (SEQ ID NO: 31).

It is understood that the spacer length will depend on the context that it is used, and that one of ordinary skill in the art will be able to ascertain the adequate length using standard techniques. In some embodiments the linker is 5-100 amino acids in length. In other embodiments the linker is 10-80, 10-70, 10-60, 10-50, 10-40, 10-30, 20-80, 20-70, 20-60, 20-50, 20-45, 20-40, 20-30, 20-25, 25-30 or 22-28 amino acids in length.

Proteolytic cleavage releases the second component of the multipart enzyme, which is then available to become sufficiently close to the first part of the multipart enzyme and generate enzymatic output. In some embodiments, the protease is a recombinant protease. In some embodiments, the protease is a tobacco etch virus (Tev) protease, an Enterokinase, or Factor Xa.

In order to direct proteolytic activity, the protease can be engineered as a fusion protein with an endogenous protein that is recruited to the receptor upon the receptors activation. In an embodiment, the protease is fused to an arrestin molecule, which is recruited to an active GPCR molecule. In an embodiment, the arrestin molecule is a β-arrestin molecule.

The system disclosed herein for monitoring receptor activation relies upon the innovation of spatially dividing two required parts of a multipart enzyme complex such that they do not interact until receptor activation occurs. The multipart enzyme complex is selected for the ability to express its components separately such that domains required for enzymatic activity are separated in the unactivated state of the system. Generally, the components of the multipart enzyme are spatially separated by engineering them such that the components are directed to different cellular compartments upon synthesis.

In some embodiments, the first component is sequestered in a cell space such as mitochondrial, endoplasmic reticulum, or cell membrane, facing the cytosolic space. In some embodiments, this sequestration is permanent, such that the first component does not exit from a particular cellular compartment. In an embodiment, the first component is sequestered to the nucleus, mitochondria, endoplasmic reticulum, or other cell membrane-integrated protein. In some embodiments, the first component is sequestered in the nucleus by linkage to a subcellular localized protein, e.g., nuclear localized protein. In some embodiments, the nuclear localized protein is Nurim (hNRM), Fibrillarin (hFBL), Transcription Factor 2 H (TFIIH; p62), Barrier to auto integration factor 1 (BANF1), or Lamin associated polypeptide 2 gamma (LAP2). In some embodiments, the first component is luxB of Photorhabdus luminescens. In some embodiments such as these cytosol-facing approaches, LuxA, when cleaved, would not need a nuclear localization sequence (NLS) to get into the nucleus. LuxA would be free to navigate the cytoplasm to find the luxB partner where it is anchored.

In some embodiments of the method, the nucleic acid encodes luxABCDEfrp and the measurable enzymatic output is bioluminescence. These embodiments utilize a lux-based bacterial luciferase reporter system. LuxA and luxB operate as a herterodimer, using luciferin (myristic aldehyde) and co-factor FMNH2 to produce bioluminescence. In addition to the luxA/B luciferase enzyme, the lux operon from the bacterium Photorhabdus luminescens produces enzymes which synthesize the myristic aldehyde substrate endogenously. This bypasses the need for an exogenous substrate used by current sensing models and greatly expand bioluminescent capabilities in a cell-based sensor scenario. Expression of the lux genes in mammalian cells enabling endogenous production of bioluminescent light has been demonstrated. In some embodiments, the nucleic acid encodes luxABCDEfrp and the measurable enzymatic output is bioluminescence. In some embodiments, the second component is luxA and the first component is luxB.

The instant disclosure contemplates harnessing lux-derived light output by linking receptor binding of a biological ligand to the production of luminescence (light). Genes encoding proteins luxCDE and frp are introduced to mammalian cells such that those proteins are constitutively produced within the cells. These proteins are responsible for the synthesis and accumulation of luciferin (myristic aldehyde) and co-factor FMNH2. By providing an internally-derived pool of substrate, the need to add substrate when taking a measurement is removed. These components are utilized by the heterodimeric enzyme luxAB to generate bioluminescence.

The receptor signaling pathway must be triggered in order for luxA and luxB to bind together, creating a bioluminescence capable enzyme. In the absence of a particular ligand, the luxA and luxB components are segregated so they cannot come together and produce light. The luxA is anchored to the receptor on the cells' surface, while luxB is anchored to the intracellular plasma membrane.

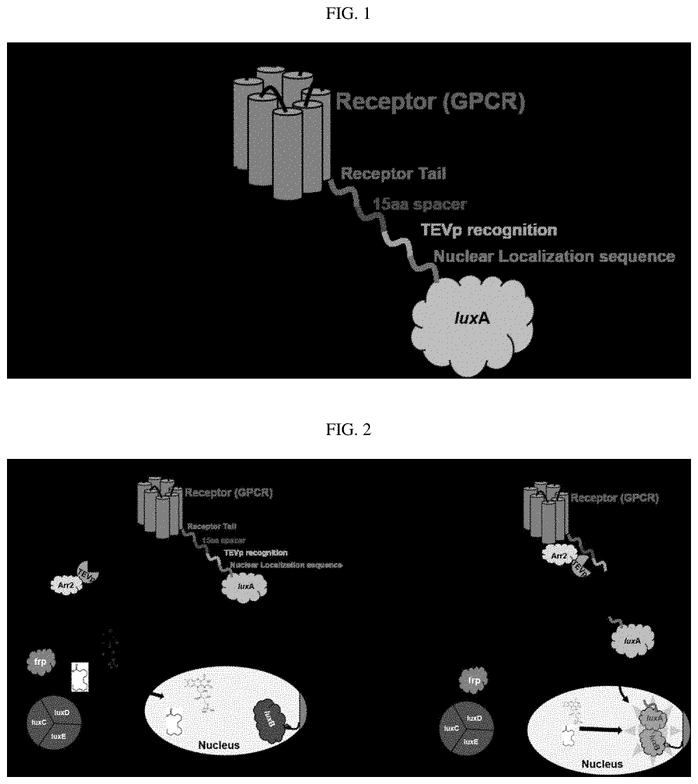

FIG. 1 is a schematic depicting exemplary components of a receptor-luxA fusion protein anchored to the cell surface. The example shows binding of the ligand to the receptor induces a natural cell transduction pathway resulting in the recruitment of an engineered arrestin2-TEV (tobacco etch virus) protease (Arr2-TEVp) to the receptor tail (FIG. 1). In binding of Arr2-TEVp to the receptor, the luxA component is freed from the receptor when TEVp recognizes its cleavage sequence (FIG. 1) and enzymatically cuts at this site. In order to access the recognition site, a 15 amino acid spacer is used between the receptor tail and TEVp cleavage site (FIG. 1). Upon release, the nuclear localization sequence attached to luxA (FIG. 1) directs its movement from the cytoplasm into the nucleus (or mitochondria, endoplasmic reticulum, or other cell membrane-integrated protein). In the nucleus, mitochondria, endoplasmic reticulum, or other cell membrane-integrated protein, the luxA can bind luxB to form an enzyme complex capable of generating bioluminescence.

FIG. 2 schematically shows transition from an unstimulated version shown in FIG. 1 to an activated system, demonstrating the receptor sensing mechanism. As described above, the second component of the enzyme is linked to a receptor protein, typically a membrane-bound receptor protein. Generally, the membrane-bound protein is a plasma-membrane-bound protein. In some embodiments, the membrane-bound receptor is a GPCR.

In an embodiment where the first component is sequestered in the nucleus, mitochondria, endoplasmic reticulum, or other cell membrane-integrated protein, the second component comprises a subcellular localization sequence. In an embodiment, the nuclear localization sequence is GRKKRRQRRR (SEQ ID NO. 32), i.e., sequences derived from HIV-TAT1 or SV40 (PKKKRKV) (SEQ ID NO: 33). Other sequences include but are not limited to proteins nucleoplasmin (AVKRPAATKKAGQAKKKKLD) (SEQ ID NO: 34), and c-Myc (PAAKRVKLD) (SEQ ID NO: 35). Numerous other such sequences are known and are included in the invention.

Generally, the nuclear localization sequence is linked to the second component such that upon protease cleavage, the nuclear localization sequence directs the second component of the multipart enzyme to the nucleus, mitochondria, endoplasmic reticulum, or other cell membrane-integrated protein.

The instant disclosure further contemplates an isolated nucleic acid encoding (a) a constitutive promoter coupled to genes encoding, (b) one or more proteins capable of producing a measurable enzymatic output, each protein comprising at least two different components, a first component anchored to a nuclear-localized protein and a second component tethered to a plasma membrane-bound receptor by a linker that comprises a protease cleavage site, wherein at least one of each of the at least two components comes together to produce enzymatic output, and (c) an enzyme complex synthesizing a substrate for the enzymatic output of the different components.

In some embodiments the nucleic acid is a plasmid comprising sensor DNA. In some embodiments the plasmid comprising sensor DNA is a plasmid comprising a nucleic acid sequence that encodes a receptor-luxA construct and a nuclear-luxB construct. In other embodiments a dual plasmid system may be used to create the cellular biosensor. For instance a cell may be transfected with two plasmids: a plasmid comprising a nucleic acid sequence that encodes a receptor-luxA construct and a plasmid comprising a nucleic acid sequence that encodes a nuclear-luxB construct.

In some embodiments an additional plasmid comprising a nucleic acid sequence that encodes a luxCDEfrp+Arr2-TEVp make be co-transfected into recombinant cells to create a cellular biosensor. This combination is particularly useful for optimizing signal to background ratio and avoiding non-specific, non-desired luminescence.

Nucleic acids can comprise deoxyribonucleic acid (DNA) or ribonucleic acid (RNA). Nucleic acids can be introduced to a target mammalian cell by any suitable means, such as transfection, i.e., lipid mediated transfection, transfection by electroporation, and viral-mediated delivery (adenovirus).

In some embodiments, the nucleic acid used to introduce the components of the system described herein is codon optimized. Codon optimization, in some embodiments, allows for translation or more efficient translation of a non-native RNA sequence. In some embodiments, the non-native RNA sequence originates from an exogenous DNA sequence introduced into the cell and transcribed by the cell's native machinery. This exogenous DNA sequence may be provided as a plasmid or other self-contained and self-maintained nucleotide vector. The exogenous DNA sequence may, in some embodiments, be integrated into the genome of the host cell.

The instant disclosure further contemplates a biological circuit, comprising: (a) a recombinant extracellular sensor, comprising a membrane-bound receptor protein, translationally fused to a first portion of a multi-part enzyme by a protease cleavable linker, wherein the first portion of the multi-part enzyme further comprises a nuclear localization sequence; (b) a second portion of the multi-part enzyme where the second portion of the multi-part enzyme is confined to the nucleus, mitochondria, endoplasmic reticulum, or other cell membrane-integrated protein; and (c) a pool of substrate for the multi-part enzyme wherein, binding of a ligand to the plasma-membrane-bound receptor results in activation of the membrane-bound receptor, cleavage of the linker at the protease cleavage site, and the first portion and the second portion of the multi-part enzyme come together, processing the pool of substrate molecules to generate a measurable signal.

In some embodiments, the biological circuit further comprises enzymes produced by exogenous nucleic acids for producing the pool of substrate. Supplying enzymes to constitutively produce substrate for the multi-part enzyme removes the need to provide exogenous substrate when detection is desired.

In an aspect, the instant disclosure relates to a method of detecting the presence or absence of a ligand or molecule as a function of receptor activation. In some embodiments, the method comprises contacting a cell with constitutively expressed nucleic acids encoding the components of the system described herein. To establish the enzymatic reporter system, a protein that produces a detectable enzymatic output is used, and constructs containing the enzymatic domains of the protein are identified, wherein each protein comprises at least two different components that can be physically separated. A first component is then engineered to be anchored to a nuclear-localized protein and a second component is engineered to be tethered to a plasma membrane-bound receptor by a linker that comprises a protease cleavage site. Binding of a ligand to the plasma-membrane-bound receptor results in activation of the receptor, cleavage of the linker by a protease, and release of the second component tethered to the receptor. In some embodiments, the method further comprises measuring levels of enzymatic output wherein detectable levels of enzymatic output indicate receptor activation.

In some embodiments, the method further comprises providing an enzyme complex that synthesizes a substrate for enzymatic output of the different polypeptide components.

In some embodiments, the method comprises taking a cell equipped with the enzymatic reporter system described herein and measuring enzymatic output. In some embodiments, the cell used herein is part of a cell line.

In some embodiments, the reporter system described herein is monitored more than once. In an embodiment, the levels of measurable enzymatic output (e.g., bioluminescence) is measured multiple times. In some embodiments, enzymatic output is measured over a period of minutes, hours, days, weeks, months, or years.

Generally, the method further comprises designing a treatment plan based upon the measured output.

### EXAMPLES

### Example 1.

Six different receptor classes were cloned into the system fused to luxA: Beta-Adrenergic 2 (ADRB2), CXC chemokine type 4 (CXCR-4), arginine vasopressin 2 (AVPR2), Thyrotropin-releasing hormone (TRHR), neuromedin B (NMBR), and Lysophosphatidic acid 1 (LPAR 1) (https://www.addgene.org/kits/roth-gpcr-presto-tango/#kit-contents). Five different nuclear-localized proteins were cloned into the system as fusions to luxB to determine which protein conformation (membrane bound, or free-floating) was optimal for reconstitution of the luxAB enzyme. The “nuclear locking” or “nuclear-localized” proteins tested were Nurim (hNRM), Fibrillarin (hFBL), Transcription Factor 2 H (TFIIH; p62), Barrier to auto integration factor 1 (BANF1), and Lamin associated polypeptide 2 gamma (LAP2).

An exemplary successful pathway initiation was demonstrated using ADRB2-luxA as the receptor fusion, and LAP2 fused to luxB, using adrenaline as the ligand/stimulant of the ADRB2 receptor. Cells expressing very high amounts of Arrestin2-TEVp demonstrated high levels of random proteolysis resulting in undesired fluorescence and luminescence in a control system, under these testing conditions (Siciliano et al Nature Communications 2018). Therefore, the amount of DNA transfected into cells was titrated in order to control expression of Arr2-TEVp and avoid non-specific signaling complications.

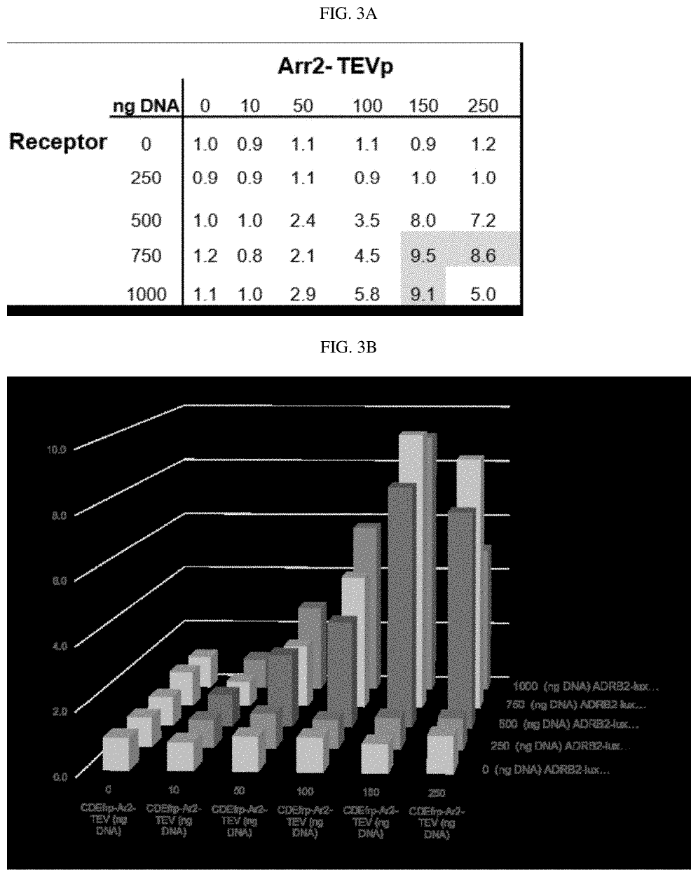

Varying amounts of sensor DNA (receptor-luxA +nuclear-luxB on one plasmid; luxCDEfrp+Arr2-TEVp on a second plasmid) were transiently transfected in order to optimize signal to background ratio and avoid non-specific, non-desired luminescence. Cells were placed in the presence and absence of 100 uM adrenaline to compare levels of bioluminescence when different amounts of DNA were present. An optimal DNA transfection ratio of between 3:1 and 5:1 (sensor : Arr2-TEVp ration of 750/150 and 750/250) provided an approximate 10:1 signal to background ratio between stimulated and unstimulated cells (FIG. 3A in tabular format and FIG. 3B in graphical representation).

### Equivalents

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1. A recombinant cell, comprising a constitutively expressed exogenous nucleic acid encoding:
(a) one or more proteins that produce a measurable enzymatic output, each protein comprising at least two different components, a first component anchored to a nuclear-localized protein and a second component tethered to a plasma membrane-bound receptor by a linker that comprises a protease cleavage site, wherein at least one of each of the at least two components comes together to produce enzymatic output; and
(b) an enzyme complex that synthesizes a substrate for enzymatic output of the different polypeptide components,
wherein, binding of a ligand to the plasma-membrane-bound receptor results in activation of the receptor, cleavage of the linker by a protease, and release of the second component tethered to the receptor.

2. The recombinant cell of claim 1, wherein the nucleic acid encodes luxABCDEfrp and the measurable enzymatic output is bioluminescence.

3. The recombinant cell of claim 1, wherein the plasma membrane protein is a G Protein-coupled receptor (GPCR).

4. The recombinant cell of claim 1, wherein the second component is luxA and the first component is luxB.

5. The recombinant cell of claim 1, wherein the linker further comprises a spacer sequence.

6. The recombinant cell of claim 1, wherein the protease is a recombinant protease.

7. A method of detecting receptor activation, comprising:
(a) introducing to a cell a constitutively expressed nucleic acid encoding:
(i) one or more proteins that produce a measurable enzymatic output, each protein comprising at least two different components, a first component anchored to a nuclear-localized protein and a second component tethered to a plasma membrane-bound receptor by a linker that comprises a protease cleavage site, wherein at least one of each of the at least two components comes together to produce enzymatic output; and
(ii) an enzyme complex that synthesizes a substrate for enzymatic output of the different polypeptide components,
wherein, binding of a ligand to the plasma-membrane-bound receptor results in activation of the receptor, cleavage of the linker by a protease, and release of the second component tethered to the receptor;
(iii) measuring levels of enzymatic output
wherein detectable levels of enzymatic output indicate receptor activation.

8. The method of claim 7, further comprising designing a treatment plan based upon the measured output.

9. The method of claim 7, wherein the constitutively expressed nucleic acid encodes luxABCDEfrp and the measurable enzymatic output is bioluminescence.

10. The method of claim 7, wherein the plasma membrane-bound receptor is a GPCR.

11. The method of claim 7, wherein the second component is luxA and the first component is luxB.

12. The method of claim 7, wherein the protease is a recombinant protease.

13. (canceled)

14. A biological circuit, comprising:
(a) a recombinant cellular sensor, comprising a membrane-bound receptor protein, translationally fused to a first portion of a multi-part enzyme by a protease cleavable linker, wherein the first portion of the multi-part enzyme further comprises a nuclear localization sequence;
(b) a second portion of the multi-part enzyme where the second portion of the multi-part enzyme is confined to the nucleus, mitochondria, endoplasmic reticulum, or other cell membrane-integrated protein; and
(c) a pool of substrate for the multi-part enzyme
wherein, binding of a ligand to the plasma-membrane-bound receptor results in activation of the membrane-bound receptor, cleavage of the linker at the protease cleavage site, and the first portion and the second portion of the multi-part enzyme come together, processing the pool of substrate molecules to generate a measurable signal.

15. The biological circuit of claim 14, further comprising enzymes, produced by exogenous nucleic acids for producing the pool of substrate.

16. (canceled)

17. A recombinant cell, comprising a constitutively active exogenous nucleic acid encoding:
(a) a recombinant sensor, comprising:
(i) a ligand binding domain;
(ii) a transmembrane domain;
(iii) a protease cleavage site;
(iv) an anchor site sequence such as a nuclear-localization sequence; and
(v) a second component of a two-part enzyme,
wherein the protease cleavage site is between the transmembrane domain and the anchor site sequence;
(b) a nuclear receiver comprising:
(i) a first component of the two-part enzyme;
(ii) a nuclear-localized protein; and
(c) an enzyme complex that produces a substrate, a co-factor, or both a substrate and a co-factor.

18. The recombinant cell of claim 17, wherein the recombinant sensor further comprises a spacer sequence between the transmembrane domain and the protease cleavage site.

19-20. (canceled)

21. A biosensor device comprising:
a housing comprising
(a) a surface for accepting a ligand,
(b) a recombinant cell within the housing having a biological circuit, wherein the biological circuit comprises a multi-part enzyme, wherein two or more parts of the multi-part enzyme have spatially distinct localizations and wherein when the recombinant cell is exposed to the ligand, the two or more parts of the multi-part enzyme are triggered to co-localize and interact with a substrate molecule to generate a measurable immediate read-out bioluminescent signal, and
(c) a surface for observing the bioluminescent signal.

22-31. (canceled)

32. A method for sensing a ligand in real-time, comprising,
exposing a putative ligand containing material to a biosensor device,
examining the biosensor device for the presence of a bioluminescent signal immediately upon exposure to the putative ligand containing material, wherein the presence of a bioluminescent signal in the biosensor device indicates the presence of the ligand in the material in real-time,
wherein the biosensor device comprises a recombinant cell having a biological circuit, wherein the biological circuit comprises a multi-part enzyme, wherein two or more parts of the multi-part enzyme have spatially distinct localizations and wherein when the recombinant cell is exposed to the ligand, the two or more parts of the multi-part enzyme are triggered to co-localize and interact with a substrate molecule to generate a measurable immediate read-out bioluminescent signal indicative of the presence of the ligand.

33. The method of claim 32, wherein the biosensor device is a device of claim 21.
